(19) United States
(12) Patent Application Publication
(10) Pub. No.: US 2026/0108003 A1
(43) Pub. Date: Apr. 23, 2026

(54) TOURNIQUET HAT ASSEMBLY

(71) Applicant: John Perkinson, Laurens, SC (US)

(72) Inventor: John Perkinson, Laurens, SC (US)

(21) Appl. No.: 19/423,518

(22) Filed: Dec. 17, 2025

Related U.S. Application Data
(63) Continuation-in-part of application No. 17/932,835, filed on Sep. 16, 2022, now abandoned.

(51) Int. Cl. A42B 1/24; A42B 1/0182; A42B 1/22; A61B 17/132

(52) U.S. Cl. CPC A42B 1/24; A42B 1/0182; A42B 1/22; A61B 17/1327

(57) ABSTRACT

A tourniquet hat assembly includes a wearable hat having an inner surface and an inner perimeter, and a tourniquet member removably retained adjacent the inner surface. A retaining structure along the inner perimeter defines a channel that receives the tourniquet member. In one embodiment, the retaining structure comprises a flexible lip forming an open-top channel that normally retains the tourniquet, but can be folded downward to expose and remove the tourniquet in an emergency. In another embodiment, the retaining structure comprises a sleeve formed by securing an upper edge of a lip to the hat to define a closed channel. The tourniquet member includes a strap and a tightening mechanism, such as a mechanical ratcheting knob or windlass rod, that can tighten the tourniquet around a limb and also adjust an effective perimeter of the hat when worn.

### CROSS-REFERENCE TO RELATED APPLICATIONS

This application is a continuation-in-part of U.S. patent application Ser. No. 17/932,835, filed Sep. 16, 2022, entitled “Tourniquet Hat Assembly,” the entirety of which is hereby incorporated by reference.

### FIELD OF THE INVENTION

The present invention relates generally to wearable tourniquet assemblies that allow a user to easily carry a tourniquet with him or her at any time. More specifically, the present invention includes a wearable hat, such as a baseball hat or any other type of hat, with a tourniquet attached to the inner liner of the hat or with the tourniquet acting as the adjustable closure of the hat. Whenever a user needs to use the tourniquet, the user simply removes the tourniquet from the inside of the hat.

### BACKGROUND OF THE INVENTION

A tourniquet is generally defined as a device for stopping the flow of blood through a vein or artery, typically by compressing a limb with a cord or tight bandage. Various types of tourniquets are commonly used in emergency situations to stop the flow of blood until a victim can receive proper medical treatment. Examples of commonly used tourniquets are as follows:

### Silicone Ring Auto-Transfusion Tourniquet

The silicone ring auto-transfusion tourniquet (SRT/ATT/EED), or surgical auto-transfusion tourniquet (HemaClear), is a simple to use, self-contained, mechanical tourniquet that consists of a silicone ring, stockinet, and pull straps that results in the limb being exsanguinated and occluded within seconds of application. The tourniquet can be used for limb procedures in the operating room, or in emergency medicine as a means to stabilize a patient until further treatment can be applied.

### Combat Application Tourniquet (CAT)

The combat application tourniquet is used by the U.S. and coalition militaries to provide soldiers a small and effective tourniquet in field combat situations. It is also used in the UK by NHS ambulance services, along with some UK fire and rescue services. The unit utilizes a windlass with a locking mechanism and can be self-applied. The CAT has been adopted by military and emergency personnel around the world.

The tourniquet strap comes completely apart to place around a limb, but it's a closed-loop system that has only a limited three-inch uptake. This means users must tightly secure the tourniquet strap to the limb prior to engaging the plastic windlass. Additionally, according to the manufacturer, for proper application the tourniquet strap must be through the plastic buckle differently depending on whether it's being used on an upper or lower extremity. The strap is secured on itself with Velcro, and there is one locking point for the windlass on this device.

### Special Operations Forces Tourniquet (SOF-T)

The SOF-T is a true open-loop tourniquet with a solid metal windlass. Early versions have a narrow strap and a metal “alligator” clip with a locking screw to secure the strap. There are two plastic D-ring securing points for the windlass once applied.

Heretofore, many efforts have been made to provide wearable tourniquets. Some examples are set forth hereinbelow, and each of the following references are incorporated herein by reference, in their entireties:

### U.S. Pat. No.4,384,583 Tourniquet

A tourniquet constructed from a length of elastic material. The ends of the elastic material are made non-elastic, and complementary coupling components are attached thereto.

The elastic material is provided with a thickening for stress absorption and control.

### U.S. Pat. No.7,604,651 Military Clothing Having Built-in Tourniquets

An article of clothing, body armor or garment having at least one limb-receiving sleeve includes a housing secured in encircling relation to the sleeve. A self-sustaining tourniquet is disposed within the housing. An opening is formed in the housing and a cover is provided to close the opening. The tourniquet is positioned fully within the housing when the cover is closed. The tourniquet is oriented in the housing so that a free end of the tourniquet is in registration with the opening. A handle is secured to the free end and is adapted to be grasped by a user when the cover is open. The diameter of the tourniquet is reduced when the handle is pulled upon. A ratchet and pawl structure prevents the tourniquet from loosening after the handle has been pulled.

### U.S. Pat. No. 8,707,468 Protective Garment With Tourniquet

A protective garment including a waist portion adapted to be donned proximate the waist of a wearer and first and second leg portions extending from a waist portion to at least proximate a knee portion of the wearer and a tourniquet member attached to an inner surface of at least one of said first and second leg portions.

### U.S. Pat. No. 9,456,826 Tourniquet Device for Medical Emergencies

A tourniquet device includes a tourniquet strap having an outward facing surface and an inward facing surface, the outward facing surface being covered with a plurality of hook fasteners, the inward facing surface having a section that is provided with a plurality of loop fasteners, a first end and a second end, the first end comprising a buckle, the second end having a cavity, and a securement strap configured for storage within the cavity, the securement strap including a terminal end that includes a pull tab, the securement strap is configured to be selectively placed in a stored position and a deployed position.

### U.S. Pub. No. US 2008/0243172 Garment With Affixed Tourniquet

The invention provides an article of clothing having a tourniquet affixed to the clothing at one end of the tourniquet, the tourniquet comprising a band comprised of an elastic and flexible material, the band having a width and a length, a first end and a second end, and a first face and second face, where at least a portion of the first face comprises hook-type fastener means, and where the second face comprises loop-type fastener means.

These prior art systems demonstrate various ways of storing or attaching tourniquets to garments, typically through rigid housings, sewn pockets, or bands integrated into clothing.

However, none of these references disclose or suggest the unique configuration of the present invention, in which a tourniquet is retained within the inner circumference of a hat in either (a) a closed sleeve formed by sewing or adhering the lip, or (b) a flexible open lip forming an open-top channel that can be folded downward for immediate removal.

In the context of integrating a tourniquet with wearable headgear such as a hat, the present inventor recognized that traditional approaches—such as fixed housings, pockets, or fully enclosed sleeves—may hinder rapid removal in an emergency. The disclosed invention therefore contemplates both (1) a fully enclosed sleeve or channel formed, for example, by stitching the upper edge of a lip to the hat's inner surface, and (2) a preferred embodiment in which a flexible lip defines an open-top channel allowing the lip to fold outward or downward to instantly expose and release the tourniquet member. These variations are further supported and illustrated in the drawing figures discussed herein.

### BRIEF SUMMARY OF THE INVENTION

In accordance with one aspect of the invention, a first embodiment of a tourniquet hat assembly includes a hat with an inner surface and an outer surface, a channel or sleeve defined about an inner perimeter of the hat, and a tourniquet member having a strap with a first end and a second end, an adjustable member, and tightening mechanism. The tourniquet member can be removably attached within the channel or sleeve of the inner surface of the hat and is positioned about the inner circumference of the hat.

Another embodiment of a tourniquet hat assembly includes the tourniquet member being removably attached to the hat, such that the tourniquet member can be used as a means for adjusting the size of the hat to accommodate the size of a user's head. The tourniquet is preferably made of a durable, washable material. The tightening mechanism is typically a durable rod-like structure (commonly referred to as a ‘windlass’ when used in context of a tourniquet) for winding the tourniquet when in use. In other embodiments, the tightening mechanism may comprise a mechanical ratcheting knob as described herein, which incrementally tightens the tourniquet member when rotated and includes a release mechanism for selectively loosening the tourniquet member.

In certain embodiments, the channel or sleeve may be implemented as a closed structure formed by sewing or adhering an upper edge of a lip portion to the inner surface of the hat, thereby creating a fully enclosed sleeve for receiving the tourniquet member. In other embodiments—including a preferred embodiment illustrated herein—the retaining structure comprises a flexible lip that extends along the inner perimeter of the hat and defines an open-top channel into which the tourniquet member may be inserted. This flexible lip may be folded downwardly or outwardly to expose the tourniquet member for rapid removal in an emergency situation, without requiring the tourniquet to be threaded through a closed sleeve.

This tourniquet/hat assembly is ideal for off-duty paramedics, military, and other rescue personnel who might need to respond to an emergency situation requiring a tourniquet. Additionally, it may be integrated into uniforms for rescue personnel to ensure they carry a tourniquet with them at all times.

By providing multiple embodiments—one employing a closed sleeve structure for secure containment, and another employing a fold-down lip structure for rapid-release functionality—the invention offers customizable performance characteristics suited to a range of applications while maintaining full compatibility with conventional hat designs and manufacturing methods.

### BRIEF DESCRIPTION OF THE DRAWINGS

These and other features, aspects, and advantages of the present invention will become better understood with regard to the following description, appended claims, and accompanying drawings where:

FIG. 1 is a perspective view of one embodiment of a hat/tourniquet assembly, showing a tourniquet positioned within the inner rim portion of the hat, and further including a tightening mechanism for adjusting the diameter of both the hat and the tourniquet; in this embodiment, the retaining structure along the inner perimeter may be implemented as either a closed sleeve or, in a preferred embodiment, as a flexible lip defining an open-top channel capable of folding downwardly to permit rapid access to the tourniquet member.

FIG. 2 is a cross-sectional view along the lines 2-2 shown in FIG. 1, wherein the tourniquet is disposed within an inner rim or channel disposed about a lower periphery of the hat; as illustrated, the channel may appear closed when the lip is in its upright position, although in preferred embodiments the lip is not sewn shut and may be folded downward as described in connection with FIG. 5-6.

FIG. 3 is a perspective view of one embodiment of a hat/tourniquet assembly, wherein the tourniquet is positioned within the inner rim of a hat, and further showing tightening mechanism including a windlass that may be twisted to tighten the tourniquet and secured in position to maintain pressure of the tourniquet about a person's limb or body member;

FIG. 4 is a perspective view of one embodiment of a hat/tourniquet assembly, wherein the tourniquet is positioned within the inner rim of a hat, and further showing a removable windlass member that is removably attached to the bill of the hat, so that both the windlass and the tourniquet may be removed from the hat in an emergency situation;

FIG. 5 is a perspective view of one embodiment of a hat/tourniquet assembly, wherein the tourniquet is removably positioned about the inner periphery or rim of the hat within a sleeve or lip positioned thereon; in a preferred configuration, the structure shown as element 20 comprises a flexible lip defining an open-top channel that retains the tourniquet during normal wear yet may be folded downward to expose the tourniquet for removal.

FIG. 6 is a perspective view of the same embodiment shown in FIG. 5, further showing the lip partially folded downwardly to create an opening for insertion or removal of the tourniquet; FIG. 6 illustrates the operation of the flexible lip embodiment, in which the lip 20 is not sewn shut and may be displaced outwardly or downwardly to permit rapid access to the tourniquet member without requiring the strap to be threaded through a closed channel.

### DETAILED DESCRIPTION OF THE INVENTION

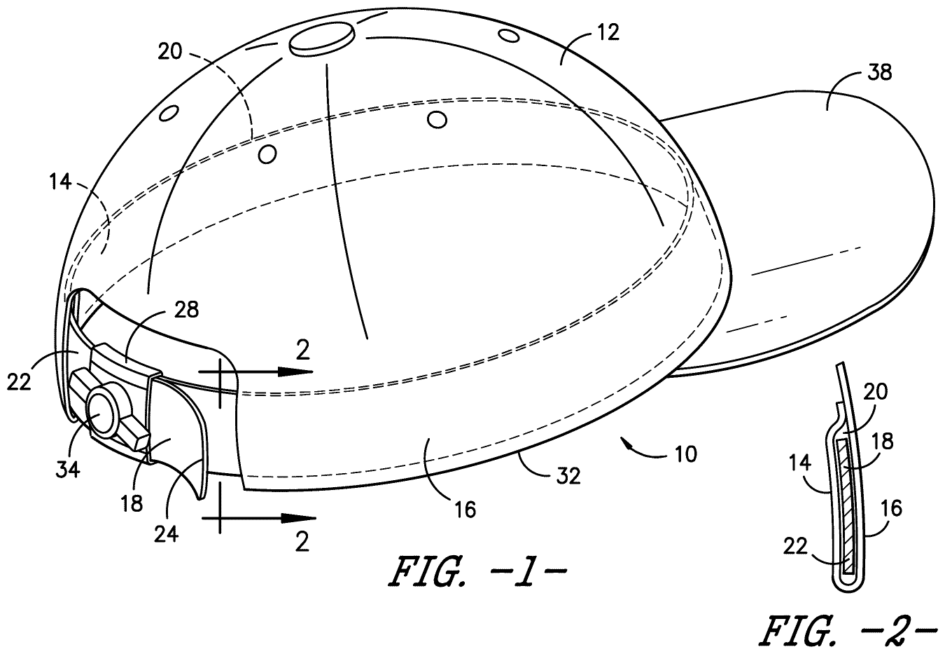

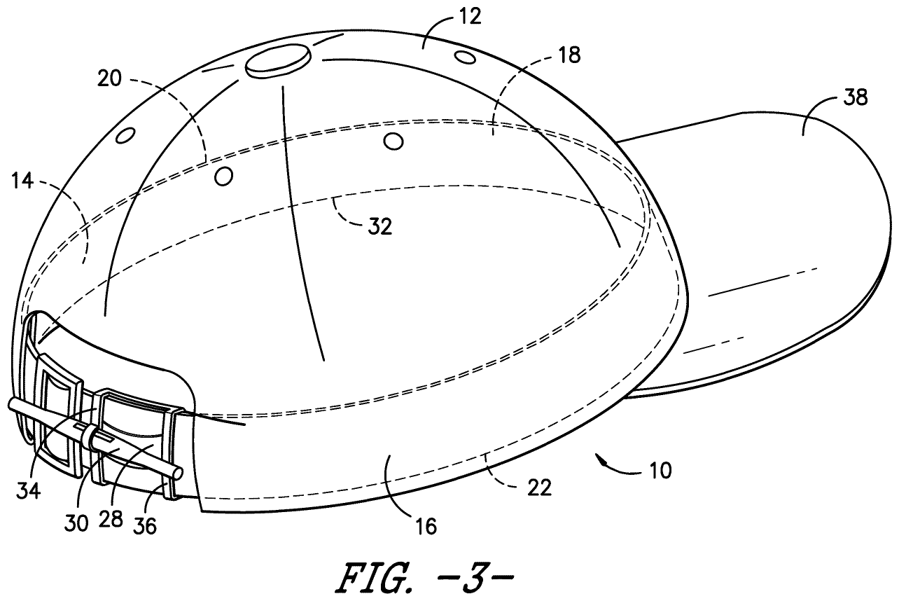

The present invention includes, in a first embodiment, a tourniquet hat assembly 10, as shown in FIGS. 1-6, and includes a baseball-style hat 12 with an inner surface 14 and an outer surface 16 and a tourniquet member 18 removably positioned within an interior portion of the hat 12, preferably within a sleeve or channel 20 about the perimeter 32 of the hat 12. The tourniquet member 18 preferably includes a strap 22 with a first end 24 and a second end 26, an adjustable member 28, and a tightening mechanism 30, such as a durable rod-like structure or the like (also referred to as a ‘windlass’ 30) or as a mechanical ratcheting knob assembly 34 as described herein. In some embodiments, the adjustable member 28 and the tightening mechanism 30 are provided by the same mechanical ratcheting knob assembly that incrementally tightens the strap 22 when rotated and includes a release mechanism for selectively loosening the strap 22; in other embodiments, the adjustable member 28 may be a buckle or similar device and the tightening mechanism 30 may comprise a windlass rod that is rotated to increase tension in the tourniquet member 18.

The tourniquet member 18 can be made from any suitable durable material. The tourniquet member 18 preferably fits inside of a channel or sleeve 20 within the inner perimeter of the hat, as shown in FIGS. 1-6, so that a user can carry the tourniquet member 18 with him or her at all times.

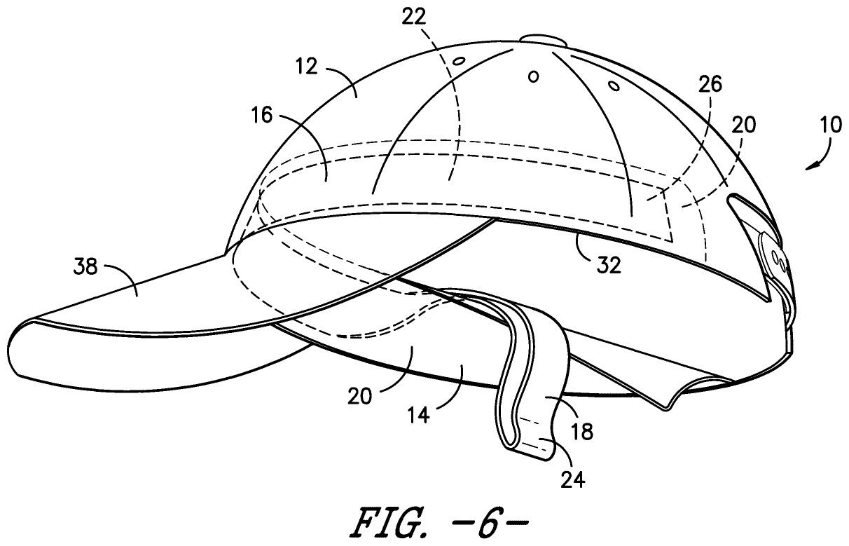

As used herein, the term “sleeve or channel 20” encompasses two structurally distinct but functionally related embodiments: (1) a closed sleeve embodiment in which the upper edge of the lip forming the sleeve is sewn, stitched, or otherwise adhered to the inner surface 14 of the hat 12, thereby creating a tubular enclosure around the perimeter 32; and (2) an open-top, flexible lip embodiment in which the retaining structure 20 extends upwardly or inwardly from the inner surface 14 to define an open channel that can be folded downwardly or outwardly to permit insertion or removal of the tourniquet member 18 without threading. The flexible lip embodiment is shown most clearly in FIGS. 5 and 6.

In a preferred embodiment, the tourniquet member 18 fits within a channel or sleeve 20 positioned on the inner perimeter 32 of the hat 12, such that the hat 12 can be worn comfortably by the wearer while the tourniquet member 18 sits therein. Traditionally, baseball-style hats include an inner perimeter 32 that includes a channel 20, lip, sleeve, or the like, which is traditionally used to carry an adjustable strap for adjusting the size of the hat 12. In one embodiment of the present invention, the tourniquet member 18 is similarly positioned within this channel 20 or sleeve, and is removable in the event of an emergency that requires a tourniquet as part of the first aid response.

Where the channel 20 is implemented as a closed sleeve, the upper edge of the lip is secured—such as by stitching, bonding, or fabric integration—to the inner surface 14 of the hat 12, thereby forming a continuous tubular pathway extending about the hat's circumference. In contrast, in the flexible-lip embodiment, the upper edge of the lip remains unattached, allowing the lip 20 to flex or fold downwardly and outwardly, as illustrated in FIGS. 5 and 6. This configuration provides rapid release of the tourniquet member 18 by simple displacement of the lip.

It should be understood that the hat 12 can be any style of hat, including, but not limited to a baseball cap, a fitted hat, a beanie, a bucket hat, or any other suitable style of hat. In order to secure the tourniquet member 18 to the inside of the hat 12, the tourniquet member 18 can be removably attached to the hat 12 within the channel 20 in the inner perimeter 32 thereof, as set forth above, or alternatively, with hook and loop fasteners, snaps, buttons, stitching, or other attachment means. When the need arises for the user to make use of the tourniquet member 18, the user may simply grab the first end 24 and pull the tourniquet member 18 out of the channel or sleeve 20 disposed about the inner periphery 32 of the hat 12 until it is removed therefrom, as shown in FIG. 5 (showing the tourniquet 18 in a partially removed state from the hat 12).

In the flexible-lip embodiment, removal is facilitated by folding the lip 20 downwardly, thereby exposing the tourniquet member 18 for withdrawal. As shown in FIG. 6, which depicts the same embodiment as FIG. 5, the lip 20 is partially folded downwardly to define an open access path. This preferred configuration avoids the need to feed or unthread the tourniquet member through a closed channel and allows substantially instantaneous deployment in emergency conditions.

In one embodiment, the tightening mechanism is a mechanical ratcheting knob 34, and is attached to the tourniquet member 18 so that it faces away from the rear of the hat 12, as shown in FIG. 1. The mechanical ratcheting knob 34, similar to those used on snowboard boots and the like, may be tightened by twisting the handle 36 in one direction. The mechanical ratcheting knob 34 may also be loosened or released, and then readjusted, and such tightening mechanisms are well known in the art (particularly as applied to tourniquets).

In one embodiment, as particularly illustrated in FIG. 1, the tightening mechanism 30 comprises a mechanical ratcheting knob 34. The mechanical ratcheting knob 34 includes a knob body 36 coupled to a length of cable, cord, or strap that forms part of the tourniquet member 18, and an internal ratchet-and-pawl assembly. Rotation of the knob body 36 in a tightening direction causes incremental uptake of the cable, cord, or strap into the tightening mechanism 34, thereby progressively tightening the tourniquet member 18 around the inner perimeter 32 of the hat 12 or around a limb when the tourniquet member 18 is deployed. During tightening, the ratchet mechanism may produce audible or tactile “clicks” as successive ratchet steps are engaged. A release mechanism, such as a push-button, pull-out, or lift-to-release actuator associated with the knob body 36, can be actuated to disengage the ratchet and allow the cable, cord, or strap to be loosened or returned toward a slack condition. After tension has been released, further rotation of the knob body 36 in the tightening direction begins a new tightening cycle from the new starting length of the tourniquet member 18. Mechanical ratcheting knob assemblies of this type are widely available and are used, for example, in footwear tightening systems and other adjustable equipment, and such mechanisms may be adapted for use in the tourniquet hat assembly 10.

Various types of tourniquets may be used, including windlass tourniquets, an example of which may be found in U.S. Pat. No. 8,343,182, which is incorporated herein by reference. For example, in one embodiment, a windlass tourniquet assembly includes an elongate main strap extending between a first end and an opposite second end, and attachment means (e.g., a buckle) for attaching the first end of the main strap to the second end of the strap. At least a portion of the attachment means is disposed at the first end of the elongate main strap. The assembly further includes a windlass strap extending between a first and second end, the first end of the windlass strap being secured to a front surface of the main strap at the first end of the main strap near the buckle or other attachment means. The second end of the windlass strap is secured to the main strap at a location a predetermined distance away from the attachment means.

In a typical windlass-type tourniquet, an elongate rod is rotated to twist at least a portion of the tourniquet strap, thereby shortening an effective length of the strap and increasing circumferential pressure when the strap is wrapped around a limb. Once adequate pressure is achieved, the elongate rod may be placed into one or more clips, cradles, or other retaining structures to prevent the rod from unwinding. As used herein, the terms “windlass rod” and “windlass crank” refer to such elongate rod-like members that are manually rotated to increase tension and tighten the tourniquet strap. In the context of the tourniquet hat assembly 10, the windlass rod may likewise be received in one or more clips, cradles, or other retaining structures carried on the tourniquet member or hat so as to maintain the tension applied to the tourniquet member until deliberate release.

Anchor means for anchoring the attachment means in a position for applying the tourniquet may be disposed on a back surface of the main strap opposite the main strap's front surface. One example of anchor means is an anchor strap including a plurality of loops formed by attaching (e.g., stitching) the anchor strap to the main strap at a plurality of locations along the length of the anchor strap. The assembly also includes an elongate windlass crank that is attachable to the anchor strap during use so that when the crank is rotated the anchor strap is twisted, effectively shortening the length of the main strap and thereby tightening the main strap (as a result of the attachment of the windlass strap to the main strap) for use as a tourniquet.

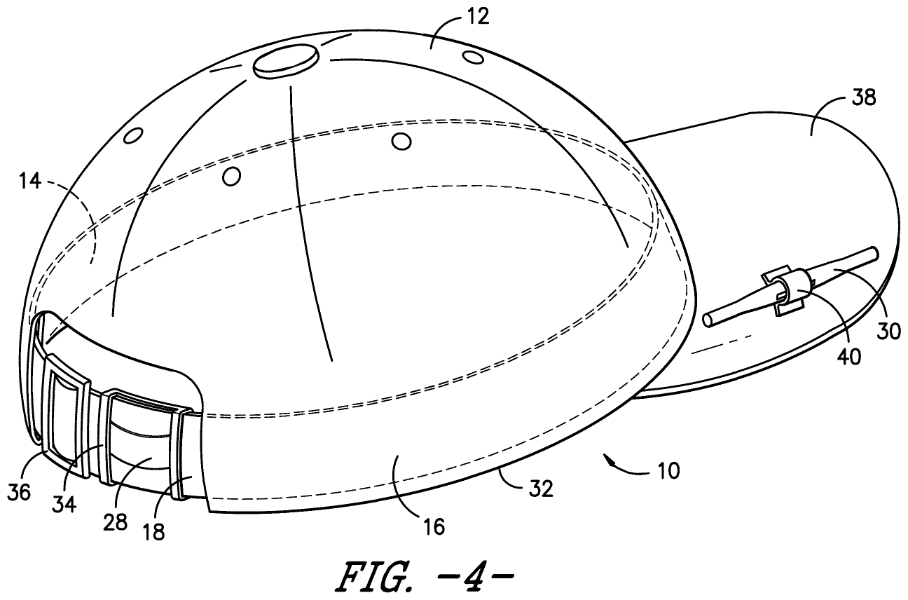

In one embodiment, the windlass crank 30 can be secured at the back portion of the hat 12 and act as an adjustable closure for the hat 12. Most baseball hats known in the art have an adjustable snapback closure or adjustable backstrap. The tourniquet member 18 fits within the hat 12 such that the windlass crank 30 of the tourniquet member 18 (or other tightening mechanism, as shown in FIG. 1) sits at the back of the hat 12 and allows the user to adjust the size of the hat 12 by rotating the windlass crank, as shown in FIG. 3. The buckle 36 may be used to secure the tightened strap 22 in position.

It is also contemplated that the tourniquet member 18 can be removed and both the hat 12 and the tourniquet member 18 can be washed. Additionally, the hat 12 can be worn without the tourniquet member 18 if the user desires.

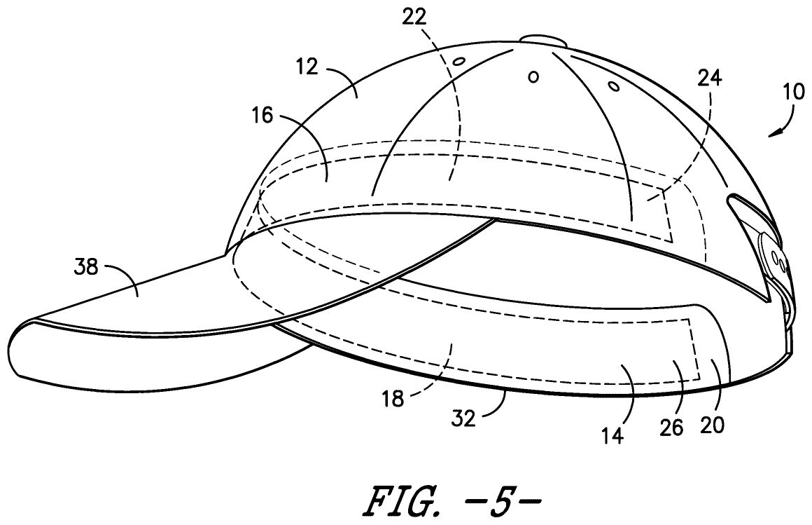

In one embodiment, as shown in FIG. 4, the windlass crank 30 is removably attached to the bill 38 of the hat 12, and may be removed therefrom, along with the tourniquet member 18, in the event of an emergency. The windlass crank 30, which may be implemented as a windlass rod as described above, may be attached to the bill 38 of the hat 12 using any suitable method, including hook and loop fasteners, a clip, a rigid loop, an elastic loop 40 that stretches and frictionally engages the windlass crank 30 when it is positioned within the elastic loop 40, or any other structure configured to removably retain the windlass rod on or adjacent the bill 38 when not in use and to permit quick removal of the windlass rod in the event of an emergency.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

1. A tourniquet hat assembly, comprising:
a wearable hat having an inner surface and an outer surface and an inner perimeter;
a tourniquet member comprising a strap with a first end and a second end and a tightening mechanism;
a retaining structure disposed along at least a portion of the inner perimeter, the retaining structure comprising a flexible lip that extends upwardly or inwardly from the inner surface and defines an open-top channel, the flexible lip being movable between an upright retaining position and a downwardly folded open position; and
wherein folding the flexible lip into the downwardly folded open position exposes the tourniquet member and permits removal of the tourniquet member from the hat, and wherein the tightening mechanism is configured to tighten the tourniquet member when the tourniquet member is applied about a limb and to adjust an effective perimeter of the hat when the hat is worn.

2. The tourniquet hat assembly set forth in claim 1, wherein the tightening mechanism comprises a windlass rod.

3. The tourniquet hat assembly set forth in claim 2, wherein the windlass rod is configured to be rotated to tighten the tourniquet member.

4. The tourniquet hat assembly set forth in claim 1, wherein the windlass rod is removably attachable to a bill of the hat when not in use.

5. The tourniquet hat assembly set forth in claim 1, wherein the windlass rod is removably attachable to the bill of the hat by an attachment selected from the group consisting of hook and loop fasteners, a clip, a rigid loop, and an elastic loop.

6. The tourniquet hat assembly set forth in claim 1, wherein the tightening mechanism comprises a mechanical ratcheting knob.

7. The tourniquet hat assembly set forth in claim 6, wherein the mechanical ratcheting knob is configured to incrementally tighten the tourniquet member when the mechanical ratcheting knob is rotated in a tightening direction, and further comprises a release mechanism configured to release tension in the tourniquet member and allow the mechanical ratcheting knob to begin a subsequent tightening cycle from a new starting length of the tourniquet member.

8. The tourniquet hat assembly set forth in claim 6, wherein the mechanical ratcheting knob is positioned at a rear portion of the hat so that adjustment of the mechanical ratcheting knob adjusts the effective perimeter of the hat about a wearer's head.

9. The tourniquet hat assembly set forth in claim 7, wherein rotation of the mechanical ratcheting knob in the tightening direction produces audible or tactile feedback indicating incremental tightening steps.

10. A tourniquet hat assembly, comprising:
a wearable hat having an inner surface and an outer surface and an inner perimeter;
a tourniquet member comprising a strap with a first end and a second end and a tightening mechanism; and
a sleeve extending along at least a portion of the inner perimeter, the sleeve being formed by securing an upper edge of a lip to the inner surface of the hat so as to define a closed channel extending about the inner perimeter;
wherein the tourniquet member is removably positioned within the closed channel of the sleeve and extends circumferentially about the inner perimeter of the hat, and wherein the tightening mechanism is configured to tighten the tourniquet member when the tourniquet member is applied about a limb and to adjust an effective perimeter of the hat when the hat is worn.

11. The tourniquet hat assembly set forth in claim 10, wherein the sleeve is formed by sewing the upper edge of the lip to the inner surface of the hat along the inner perimeter.

12. The tourniquet hat assembly set forth in claim 10, wherein the tourniquet member is removable from the closed channel by pulling the first end of the strap from the sleeve.

13. The tourniquet hat assembly set forth in claim 10, wherein the hat is selected from the group consisting of a baseball cap, a fitted hat, a bucket hat, a beanie, and a military cap.

14. The tourniquet hat assembly set forth in claim 10, wherein the tightening mechanism comprises a mechanical ratcheting knob that is positioned at a rear portion of the hat so that adjustment of the mechanical ratcheting knob adjusts the effective perimeter of the hat about a wearer's head.

15. The tourniquet hat assembly set forth in claim 10, wherein the tightening mechanism comprises a windlass rod configured to be rotated to tighten the tourniquet member and securable in a retaining structure to maintain tension in the tourniquet member.

16. A method for providing a tourniquet hat assembly, comprising the steps of:
providing a hat having an inner surface, an outer surface, and an inner perimeter;
providing a tourniquet member having a first end and a second end and a tightening mechanism;
providing a retaining structure disposed along at least a portion of the inner perimeter of the hat and configured to receive the tourniquet member; and
positioning the tourniquet member adjacent the inner surface of the hat such that the tourniquet member is disposed about the inner perimeter of the hat and is retained within the retaining structure;
wherein the retaining structure comprises at least one of:
(i) a flexible lip that extends upwardly or inwardly from the inner surface and defines an open-top channel; and
(ii) a sleeve extending along at least a portion of the inner perimeter and formed by securing an upper edge of a lip to the inner surface of the hat to define a closed channel.

17. The method set forth in claim 16, wherein the retaining structure comprises the flexible lip that defines the open-top channel, and further comprising the step of configuring the flexible lip so that it is movable between an upright retaining position and a downwardly folded open position to expose the tourniquet member for removal from the hat.

18. The method set forth in claim 16, wherein the retaining structure comprises the sleeve that defines the closed channel, and further comprising the step of inserting the tourniquet member into the closed channel so that the tourniquet member extends circumferentially about the inner perimeter of the hat.

19. The method set forth in claim 16, further comprising the step of positioning the tightening mechanism at a rear portion of the hat so that operation of the tightening mechanism adjusts an effective perimeter of the hat about a wearer's head.

20. The method set forth in claim 16, further comprising the step of attaching the tightening mechanism, when not in use as a tourniquet, to a bill of the hat by an attachment selected from the group consisting of hook and loop fasteners, a clip, a rigid loop, and an elastic loop.
